(19) United States
(12) Patent Application Publication
(10) Pub. No.: US 2026/0241385 A1
(43) Pub. Date: Aug. 20, 2026

(54) HEATING SYSTEM AND METHOD

(71) Applicant: LumiraDx UK Ltd., London (GB)

(72) Inventor: David William Taylor, Alloa (GB)

(21) Appl. No.: 19/164,162

(22) PCT Filed: Mar. 11, 2024
(86) PCT No.: PCT/GB2024/050653

§ 371 Date: Sep. 10, 2025

(30) Foreign Application Priority Data
Mar. 13, 2023 (GB) 2303651.0

(51) Int. Cl. B01L 7/00; B01L 3/00; H05B 1/02

(52) U.S. Cl. CPC B01L 7/52; B01L 3/502715; H05B 1/0244; B01L 2200/025; B01L 2300/12; B01L 2300/1827; B01L 2400/06

(57) ABSTRACT

A heating system suitable for heating a region of a microfluidic device includes an RC circuit, a DC power supply delivering a constant potential V_{PS}, and a switch system. In use, the heating system alternatingly charges the capacitor of the RC circuit essentially to V_{PS}, e.g., to at least about 0.98×V_{PS}, with a first polarity and then charges the capacitor to essentially V_{PS} with a second polarity opposite the first polarity. During each charging period, the resistor of the RC circuit dissipates an amount of energy essentially given by 0.5×C×(2×V_{PS})². The heating system efficiently converts the maximum electrical potential difference generated by the DC power supply (V_{PS}) to thermal energy, i.e., heat.

### FIELD OF THE INVENTION

The present invention relates to systems and methods for generating heat such as to heat an object and/or composition of matter.

### RELATED APPLICATION

This application claims the benefit of and priority to UK Patent Application no. 2303651.0, titled “Heating System and Method, and filed 13 Mar. 2023, the entire contents of which are incorporated by reference herein. U.S. patent application Ser. No. 17/860,933 titled “Fluid Control in Microfluidic Devices” and filed Jul. 8, 2022 (“the '933 Application”) is incorporated herein in its entirety.

### BACKGROUND OF THE INVENTION

Microfluidic devices, e.g., microfluidic strips, such as disclosed in the '933 Application may be used to determine the presence and/or amount of one or more targets in a liquid sample. Typically, such microfluidic devices define a microfluidic network therein having an application zone for introducing a sample and one or more channels and chambers through which the sample is moved. The microfluidic network may include reagents configured to react with the one or more targets to facilitate the determination thereof. The microfluidic network may include valves configured to permit or obstruct the passage of sample and/or gases through portions of the microfluidic network. Other components of microfluidic networks may include sources of gas pressure configured to move samples therein.

In some cases, the reaction of samples with reagents, the operation of valves, or the gas pressure-induced movement of samples may be facilitated or accomplished by heating a portion of the microfluidic network. For example, the temperature of a mixture of sample and one or more reagents may be raised above ambient temperature to facilitate one or more reactions. A valve may include a temperature sensitive material that is heated to change the shape or phase of the material thereby opening or closing the valve. Sample movement can be induced by heating a volume of gas within a chamber of a microfluidic device thereby increasing the pressure of the gas acting on the sample.

The operation of such thermally actuated microfluidic components and processes would be improved by a heating system capable of delivering a known and reproducible amount of energy and/or power to a location of a microfluidic device associated with the component or process.

### SUMMARY OF THE INVENTION

In embodiments, a heating system includes an electrical circuit comprising first and second circuit leads and a resistive element and a capacitive element in between the first and second circuit leads, e.g., in series between the first and second circuit leads, a power supply comprising first and second supply leads and being configured to provide, e.g., generate and maintain, an electrical potential V_{PS} across the first and second supply leads, a switch system having (1) a first position in which the first supply lead is in electrical communication with the first circuit lead and the second supply lead is in electrical communication with the second circuit lead and (2) a second position in which the first supply lead is in electrical communication with the second circuit lead and the second supply lead is in electrical communication with the first circuit lead, and a switch system controller (controller) configured to cause the switch system to alternate between the first and second positions. The passage of current through the resistive element generates heat, which may be used to heat an object and/or gas or portion of an object and/or gas disposed in thermal communication with the resistive element. For example, the resistive element may be disposed in thermal communication with a region of a microfluidic device, such as a reaction chamber, detection chamber, valve, and/or gas chamber of the microfluidic device.

In any embodiments of the heating system, the power supply may be a DC power supply. The electrical potential V_{PS} may be a constant, e.g., DC, electrical potential. For example, the power supply may be configured to provide, e.g., generate and maintain, the electrical potential V_{PS} across the first and second supply leads with a relative variation as compared to the average electrical potential of less than about 10%, less than about 5%, less than about 2.5%, or about 1% or less. For example, the power supply may be configured to provide, e.g., generate and maintain, the electrical potential V_{PS} at an essentially constant electrical potential V_{PS} across the first and second supply leads.

In any of the embodiments of the heating system, at least the power supply and the switch system may be implemented as one or more integrated circuits, such as an integrated circuit(s) including two or more ½H-bridge drivers or at least one H-bridge driver. A switch system typically includes at least one switch, e.g., at least first and second switches. In any such embodiments, the controller may also be implemented as an integrated circuit, which may also include the power supply and/or the switch system.

In embodiments of the heating system, the resistive element consists essentially of a single resistive element, e.g., a single resistor, having a resistance R. In other embodiments, the resistive element includes two or more resistive elements, e.g., two or more resistors, arranged to provide a total resistance R. Exemplary resistive elements include carbon ink-based resistive elements, silver ink-based resistive elements, metal oxide, e.g., silver oxide, resistive elements, and combinations thereof. The resistive element(s) may be formed using printing technologies such as ink jet printing and screen printing.

In embodiments of the heating system, the capacitive element consists essentially of a single capacitive element, e.g., a single capacitor, having a capacitance C. In other embodiments, the capacitive element includes two or more capacitive elements, e.g., two or more capacitors, arranged to provide a total capacitance C. In any of the embodiments, the capacitive element may be configured as any of the capacitive elements disclosed herein. For example, the capacitive element(s) may be a non-polarized capacitor having a low temperature coefficient such that the capacitance exhibits low variation as a function of the temperature of the capacitor. Exemplary capacitive elements, individually or in combination, may be formed, e.g., of polyester, polypropylene, ceramic, and combinations thereof. Other suitable capacitive elements include capacitive elements, e.g., capacitors, formed by printing techniques as disclosed herein.

In any of the embodiments of the heating system, the resistive element(s) may have a total resistance R, the capacitive element(s) may have a total capacitance C, and the electrical circuit has an RC time constant given by the product R×C. The RC time constant is typically between about 1 ms and about 1000 ms, between about 1 ms and about 100 ms, between about 3 ms and 50 ms, between about 5 ms and about 25 ms, or between about 7.5 and about 15 ms. The resistance R of the RC circuit may be essentially determined by the resistance of the resistive element(s), e.g., the RC circuit may exclude other sources of resistance that contribute substantially to the total resistance of the RC circuit. The magnitude of stray resistance of the RC circuit may be small relative to the magnitude of the resistance R. For example, the ratio of R to the total resistance of the RC circuit may be at least about 0.9, at least about 0.95, at least about 0.975, or may be essentially 1.0. The capacitance of the RC circuit may be essentially determined by the capacitance of the capacitive element(s), e.g., the RC circuit may exclude other sources of capacitance that contribute substantially to the capacitance of the RC circuit. The magnitude of stray capacitance of the RC circuit may be small relative to the magnitude of the capacitance C. For example, the ratio of C to the total capacitance of the RC circuit may be at least about 0.9, at least about 0.95, at least about 0.975, or may be essentially 1.0.

In any of the embodiments of the heating system, the controller may be configured to (i) cause the switch system to switch to the first position and to maintain the switch system in the first position for a time T₁ₛₜ before causing the switch system to switch to the second position, (ii) maintain the switch system in the second position for a time T_{2nd}, and (iii) repeat steps (i) and (ii). The duration of T₁ₛₜ and T_{2nd} are each typically between about RC and 20×RC, between about 2×RC and about 10×RC, between about 3×RC and about 7×RC, e.g., about 4×RC, wherein RC is the RC time constant of the RC circuit. The durations of T₁ₛₜ and T_{2nd} may be essentially the same. The controller may be configured to repeat step (iii) a total number of N times per second, wherein N is between about (0.5×RC)⁻¹ and about (40×RC)⁻¹, e.g., about (8×RC)⁻¹. In embodiments, for example, N may be between about 5 and 500, between about 10 and 200, or between about 15 and 100.

In repeating step (iii) for a number N times per second, the average power dissipated by the resistive element typically depends on the square of twice the input potential V_{PS}, i.e., about 0.5×C×(2×V_{PS})²/τ_{P}=2×C×(V_{PS})²/τ_{P} where τ_{P} is the average of periods T₁ₛₜ and T_{2nd}. The heating system efficiently converts the difference between the maximum and minimum electrical potential generated by the power supply (V_{PS}) to energy and power dissipated by the resistive element. The energy and power dissipated are essentially independent of the resistance R.

In any of the embodiments of the heating system, the controller may be configured to (i) place the switch system in the first position for a period of time sufficient to charge the capacitive element to a potential of at least about 0.5×V_{PS}, at least about 0.80×V_{PS}, at least about 0.90×V_{PS}, at least about 0.95×V_{PS}, or at least about 0.98×V_{PS} and with a first polarity, (ii) cause the switch system to switch to the second position and to maintain the switch system in the second position for a time sufficient to discharge the capacitive element and then charge the capacitive element to a potential of at least about 0.5×V_{PS}, at least about 0.80×V_{PS}, at least about 0.90×V_{PS}, at least about 0.95×V_{PS}, or at least about 0.98×V_{PS}, with a second polarity opposite to the first polarity, (iii) cause the switch system to switch to the first position and to maintain the switch system in the first position for a time sufficient to discharge the capacitive element and then charge the capacitive element to a potential of at least about 0.5×V_{PS}, at least about 0.80×V_{PS}, at least about 0.90×V_{PS}, at least about 0.95×V_{PS}, or at least about 0.98×V_{PS}, with the first polarity, and (iv) repeat steps (ii) and (iii). The controller may be configured to repeat step (iv) a total number N times per second, wherein N is between about (0.5×RC)⁻¹ and about (40×RC)⁻¹, e.g., about (8×RC)⁻¹. In embodiments, for example, N may be between about 5 and 500, between about 10 and 200, or between about 15 and 100.

In embodiments, a heating method includes generating heat by using any of the foregoing heating systems The source of heat includes, e.g., essentially arises from, the power dissipated over the resistive element(s) of the RC circuit. In performing the heating method, the resistive element is disposed in thermal communication with an object and/or gas or region to be heated. For example, the heating method may include heating a region of a microfluidic device, with the resistive element being disposed in thermal communication with such region. Such regions may include, for example, a reaction chamber, detection chamber, valve, and/or gas chamber of the microfluidic device. In any embodiments of the heating method, the resistive element and/or capacitive element may be configured as any of the resistive elements and/or capacitive elements disclosed herein. The heating method may include any of the embodiments of heating system disclosed herein.

In embodiments, the heating method includes providing an electrical circuit comprising first and second circuit leads and a resistive element and a capacitive element between the first and second circuit leads. Typically, the resistive element and capacitive element are arranged in series. The method includes providing a power supply having first and second supply leads and configured to provide, e.g., generate and maintain, an electrical potential V_{PS} therebetween. The method further includes, alternatingly, (1) connecting the first supply lead to the first circuit lead and the second supply lead to the second circuit lead and (2) connecting the second supply lead to the first circuit lead and the first supply lead to the second circuit lead.

In any embodiments of the heating method, the power supply may be a DC power supply. The electrical potential V_{PS} may be a constant, e.g., DC, electrical potential. The method may include providing, e.g., generating and maintaining, the electrical potential V_{PS} with a relative variation as compared to the average electrical potential of less than about 10%, less than about 5%, less than about 2.5%, or about 1% or less. For example, the method may include providing, e.g., generating and maintaining, the electrical potential V_{PS} at an essentially constant electrical potential V_{PS}.

In performing any of the embodiments of the heating method, the resistive element may have a resistance R, the capacitive element may have a capacitance C, and the electrical circuit may have an RC time constant R×C. The method may include performing step (1) and maintaining the connecting for a duration T₁ₛₜ, performing step (2) and maintaining the connecting for a duration T_{2nd}, and sequentially repeating steps (1) and (2). The duration of T₁ₛₜ and T_{2nd} are each typically between about RC and 20×RC, between about 2×RC and about 10×RC, between about 3×RC and about 7×RC, e.g., about 4×RC, wherein RC is the RC time constant of the RC circuit. The durations of T₁ₛₜ and T_{2nd} may be essentially the same. The controller may be configured to repeat each of steps (1) and (2) a total number N times per second. Number N is typically between about (5×RC)⁻¹ and about (0.5×RC)⁻¹, e.g., about (2×RC)⁻¹. In embodiments, for example, N may be between about 10 and 500, between about 25 and 250, between about 50 and 200, e.g., about 70.

Performing the heating method may include (i) performing step (1) and maintaining the connecting for a period of time sufficient to charge the capacitive element to a potential of at least about 0.5×V_{PS}, at least about 0.80×V_{PS}, at least about 0.90×V_{PS}, at least about 0.95×V_{PS}, or at least about 0.98×V_{PS} and with a first polarity, (ii) performing step (2) and maintaining the connecting for a period of time sufficient to discharge the capacitive element and then charge the capacitive element to a potential of at least about 0.5×V_{PS}, at least about 0.80×V_{PS}, at least about 0.90×V_{PS}, at least about 0.95×V_{PS}, or at least about 0.98×V_{PS} and with a second polarity opposite to the first polarity, (iii) performing step (1) and maintaining the connecting for a time sufficient to discharge the capacitive element and then charge the capacitive element to a potential of at least about 0.5×V_{PS}, at least about 0.80×V_{PS}, at least about 0.90×V_{PS}, at least about 0.95×V_{PS}, or at least about 0.98×V_{PS}, with the first polarity, and (iv) repeating steps (ii) and (iii).

In embodiments, a heating method includes providing an electrical circuit comprising first and second circuit leads and a resistive element and a capacitive element arranged in series between the first and second circuit leads; providing a power supply having first and second supply leads and generating a potential V_{PS} therebetween; and alternatingly performing the following steps (1) and (2): (1) (a) flowing electrons out of the first circuit lead and into the first supply lead and (b) flowing electrons out of the second supply lead and into the second circuit lead, thereby charging the capacitive element to a potential V₁ with a first polarity and wherein a ratio V₁/V_{PS} is between about 0.5 and 1, e.g., between about 0.8 and about 1.0, between about 0.90 and about 1.0, between about 0.95 and about 1.0, or between about 0.98 and about 1.0; and (2) (a) flowing electrons out of the second supply lead and into the first circuit lead and (b) flowing electrons out of the second circuit lead and into the first supply lead, thereby charging the capacitive element to a potential V₂ with a second polarity opposite to the first polarity and wherein a ratio V₂/V_{PS} is between about 0.5 and about 1.0, e.g., between about 0.8 and about 1.0, between about 0.90 and about 1.0, between about 0.95 and about 1.0, or between about 0.98 and about 1.0. In performing the heating method, the resistive element may have a resistance R, the capacitive element may have a capacitance C, and the electrical circuit may have an RC time constant R×C. The method may include performing step (1) and maintaining the flow of electrons for a duration T₁ₛₜ, and then performing step (2) and maintaining the flow of electrons for a duration T_{2nd}, and alternatingly repeating steps (1) and (2). The duration of T₁ₛₜ and T_{2nd} are each typically between about RC and 20×RC, between about 2×RC and about 10×RC, between about 3×RC and about 7×RC, e.g., about 4×RC, wherein RC is the RC time constant of the RC circuit. The durations of T₁ₛₜ and T_{2nd} may be essentially the same. The controller may be configured to repeat steps (1) and (2) a total number N times per second, wherein N is between about (0.5×RC)⁻¹ and about (40×RC)⁻¹, e.g., about (8×RC)⁻¹. In embodiments, for example, N may be between about 5 and 500, between about 10 and 200, or between about 15 and 100.

In embodiments, a heating method includes providing an electrical circuit comprising first and second circuit leads and a resistive element(s) and a capacitive element having a capacitance C arranged in series between the first and second circuit leads; performing repeated cycles each comprising (i) charging the capacitor to a potential V with a first polarity by passing electrical current through the electrical circuit in a first direction and (ii) charging the capacitive element to the potential V with a second polarity opposite to the first polarity by passing electrical current through the electrical circuit in a second direction opposite to the first direction, thereby dissipating an amount of energy in the resistive element equal to about C×(2×V)² during each cycle. A difference between a maximum electrical potential and a minimum electrical potential applied to the first and second leads may be no greater than about 1.2×V, no greater than about 1.1×V, no greater than about 1.05×V, e.g., is about V. The absolute value of the electrical potential applied to the first and second circuit leads relative to the average electrical potential applied to the first and second leads may vary by about 20% or less, about 15% or less, about 10% or less, about 5% or less, or about 2.5% or less.

In embodiments, a self-contained diagnostic reader may include any of the foregoing embodiments of the heating system and may be configured to perform any embodiments of the heating method. The self-contained diagnostic reader is configured to operate a diagnostic device, such as a microfluidic device, e.g., a microfluidic strip or cartridge, to perform a diagnostic assay(s) on a liquid sample applied to the diagnostic device. The self-contained diagnostic reader includes the resistive element positioned so that the resistive element is in thermal communication with a region of the microfluidic device when the device is disposed in an operative position with respect to the reader. For example, the self-contained diagnostic reader may include a platen incorporating the resistor. A diagnostic device inserted in an operative position with respect to the self-contained diagnostic reader is disposed in thermal communication with the platen. During operation, the self-contained diagnostic reader operates the heating system to dissipate heat over the resistor and thereby heat the region of the diagnostic device. For example, the region of the diagnostic device may include, a reaction zone, detection zone, a valve, and/or a gas-pressure chamber, e.g., a gas-pressure chamber configured to move liquids and/or gases within the device. An exemplary valve includes a temperature sensitive material that is heated to change the shape or phase of the material thereby opening or closing the valve. In a reaction chamber, the temperature of a mixture of sample optionally including one or more reagents may be raised above ambient temperature to facilitate one or more reactions and/or mixing. A volume of gas disposed in a gas chamber may be heated thereby increasing the pressure of the gas and inducing movement of a sample or other material in gaseous communication therewith.

In embodiments, a diagnostic system may include any of the foregoing embodiments of the heating system and may be configured to perform any embodiments of the heating method. The diagnostic system includes a diagnostic device, such as a microfluidic device, e.g., a microfluidic strip, and a diagnostic reader configured to operate the diagnostic device to perform a diagnostic assay(s) on a liquid sample applied to the diagnostic device. For example, the diagnostic assay may be performed to determine the presence and/or amount of one or more target(s) present in the liquid sample. The diagnostic reader of the diagnostic system typically includes the power supply, switch system (including the at least one switch, e.g., first and second switches, and switch system controller), and controller of the heating system. The microfluidic device may include the resistive element, e.g., resistor, and/or the capacitive element, e.g., capacitor forming the RC circuit as disclosed herein. When the microfluidic device is disposed in operable position with the diagnostic reader, the supply leads of the power supply are disposed in electrical communication with the circuit leads of the RC circuit of the microfluidic device as disclosed herein. During operation of the microfluidic device, the power supply, switch system, and controller of the diagnostic reader operate to heat the resistive element as disclosed herein.

The diagnostic device, e.g., microfluidic strip or cartridge, may include the resistive element. For example, the resistive element may be disposed on a surface of the diagnostic device in thermal communication with the region to be heated, e.g., a reaction zone, a valve, or a gas-pressure chamber, e.g., a gas-pressure chamber configured to move liquids and/or gases within the device. For example, the resistive element may be adhered to the surface of the diagnostic device in thermal communication with the region to be heated. The surface may be an external surface, e.g., a surface which is not in direct contact with liquids or other materials that are manipulated within the diagnostic device. Such external surface may be disposed, e.g., between layers of substrate forming the microfluidic device. Alternatively, at least a portion, e.g., all, of the resistive element(s) may be disposed within the diagnostic device in direct contact with liquids or other materials within the diagnostic device. The resistive element may include an electrically insulating layer, e.g., a coating, to prevent shorting in the presence of liquids, other materials, or other electrical elements of the diagnostic device. Exemplary resistive elements suitable for use with a diagnostic device include carbon-based resistive elements, e.g., carbon ink-based resistive elements, silver-ink based resistive elements, metal oxide, e.g., silver oxide, resistive elements, and combinations thereof. Such resistive elements may be, e.g., printed or otherwise deposited on the diagnostic device. The diagnostic device and diagnostic reader may be configured and include features of microfluidic strips and readers disclosed in the '933 Application. The region of the diagnostic device to be heated may include, e.g., a reaction zone, detection zone, a valve, and/or a gas-pressure chamber, e.g., a gas-pressure chamber configured to move liquids and/or gases within the device.

The diagnostic device, e.g., microfluidic strip or cartridge, may include the resistive element and the capacitive element. For example, the capacitive element may be configured as a printable capacitor on a surface of the diagnostic device in electrical communication with the resistive element to form the RC circuit. The surface may be an external or internal surface of the microfluidic device. In such embodiments, the RC circuit may be disposed entirely on and/or within the microfluidic device, e.g., between layers of substrate forming the microfluidic device. The circuit leads of such RC circuit may be formed as contacts arranged to be in electrical communication with supply leads of the diagnostic reader when the microfluidic device is disposed in an operable position with respect to supply leads of the diagnostic reader. The diagnostic reader includes the power supply and switch system.

A diagnostic method may include any of the foregoing heating methods and may utilize, e.g., the self-contained diagnostic reader or the diagnostic system. The diagnostic method may include disposing a liquid sample within a reaction zone and/or detection zone of a microfluidic device, the reaction zone being in thermal communication with the resistive element and performing the heating method to heat the liquid sample within the reaction zone and/or detection zone. The diagnostic method may alternatively or in combination include heating a thermally actuated valve within a microfluidic device thereby opening or closing the valve. The diagnostic method may alternatively or in combination include heating a gas chamber within a microfluidic device so as to exert a motive pressure on a liquid and/or other material within a microfluidic network of the microfluidic device (or all three).

In embodiments, a heating method includes using any of the foregoing heating systems, including any diagnostic reader incorporating such heating systems or diagnostic systems incorporating such heating systems, to heat a composition of matter, article of manufacture, or combination thereof. For example, the heating method may be included within a diagnostic method including the determination of the presence and/or amount of one or more targets within a sample. The heating method may include, for example, heating a thermally actuated valve to open or close the valve, heating a sample, optionally including one or more reagents in combination therewith, to facilitate a reaction or mixing of the sample and/or reagent(s), and/or heating a volume of gas such as to exert a motive pressure on a liquid or other material to be moved within a microfluidic network of a diagnostic device.

In any embodiment of the heating system, heating methods, self-contained diagnostic readers, and diagnostic readers and systems and methods incorporating such heating systems and methods, the power supply may include a single source of electrical potential that is applied to the RC circuit of the heating system. Such a system would not include, for example two or more sources of electric potential alternatingly used to apply electrical potential to the RC circuit.

In any embodiment of the heating system, heating methods, and diagnostic readers and systems and methods incorporating such heating systems and methods, the electrical potential provided by the power system, e.g., V_{PS}, may be, e.g., between about 5 V and about 50 V, e.g., about 12 V.

### BRIEF DESCRIPTION OF THE DRAWINGS

FIG. 1 is a schematic illustrating a heating system of the present invention;

FIG. 2 illustrates the time dependence of an electrical potential applied to an RC circuit of the heating system of FIG. 1 during the operation thereof and, in response to the applied electrical potential: (i) the time dependence of an electrical potential drop across a resister of the RC circuit and (ii) the time dependence of an electrical potential drop across a capacitor of the RC circuit;

FIG. 3 illustrates an exemplary output of a prior art alternating current power supply in the form of a square wave oscillating between a maximum potential V and a negative potential-V;

FIG. 4 illustrates an exemplary implementation of the heating system of FIG. 1 for heating a reaction zone of a microfluidic device; and

FIG. 5 illustrates a temperature vs time plot obtained for each of two resistors using a heating system of the invention and as described in the Example; each row of the chart represents a 5° C. temperature difference and each column of the chart represents a 30 s time interval.

### DETAILED DESCRIPTION

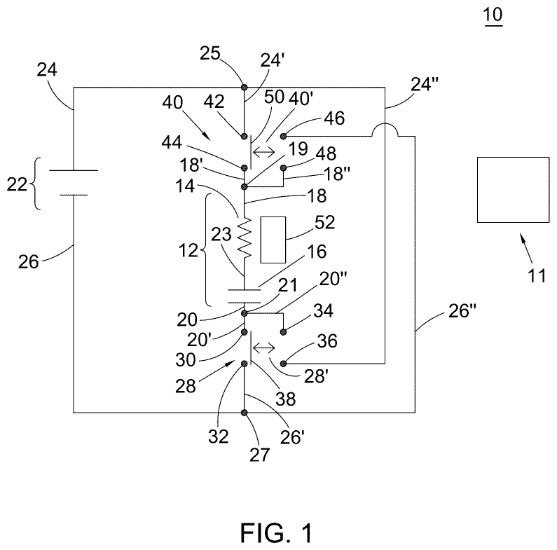

With reference to FIG. 1, a heating system 10 includes an RC circuit 12, a DC power supply 22, a first switch 28, and a second switch 40. A controller 11, e.g., an electronic processor, is configured to operate DC power supply 22 and first and second switches 28, 40. Controller 11 may be in direct electrical and/or wireless communication with heating system 10. RC circuit 12 includes a resistor 14 and a capacitor 16 arranged in series between a first RC circuit lead 18 and a second RC circuit lead 20. DC power supply 22 and first and second switches 28, 40 may be implemented using, e.g., an integrated circuit such as one providing at least two ½ H-bridge drivers or at least one H-bridge driver. An exemplary integrated circuit is the DRV8844PWP quad ½ H-bridge driver manufactured by Texas Instruments.

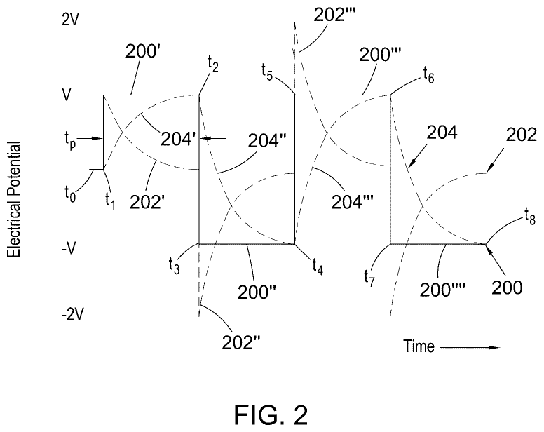

In use, resistor 14 is disposed in thermal communication with a location 52 to be heated. Location 52 may be, e.g., a region of a microfluidic network of a microfluidic device, such as a thermally controlled valve, source of gas-pressure (e.g. a gas chamber), or reaction chamber (e.g. a reaction zone) thereof. Controller 11 actuates DC power supply 22 and first and second switches 28, 40 thereby applying a time-dependent electrical potential 200 across RC circuit 12 (FIG. 2). The application of time-dependent electrical potential 200 repeatedly charges and discharges capacitor 16 with all of the charging and discharging current flowing through resistor 14. As discussed below, the current flow through resistor 14 dissipates a known amount of energy at a known rate, i.e., a known power, through resistor 14 thereby providing controllable heating of location 52. The heating can also be efficient because the heating system 10 can maximize the relative amount of electrical potential difference generated by power supply 22 that is converted to thermal energy, i.e., heat.

Resistor 14 has a resistance R. In an exemplary embodiment in which the heating system 10 is used to heat a region of a microfluidic network of a microfluidic device, R is, e.g., between about 25Ω and about 500Ω, e.g., about 150Ω. Capacitor 16 has a capacitance F. In such exemplary embodiment, F is, e.g., between about 20 μF and about 100 μF, e.g., about 50 μF. RC circuit 12 is typically formed so that resistance R of resistor 14 accounts for substantially all, e.g., essentially all, of the resistance of circuit 12 and so that the capacitance F of capacitor 16 accounts for substantially all, e.g., essentially all, of the capacitance of circuit 12 when determined at frequencies of less than 200 Hz. Thus, the magnitude of stray resistance of the RC circuit is typically small relative to the magnitude of the resistance R. For example, the ratio of R to the total resistance of the RC circuit may be at least about 0.9, at least about 0.95, at least about 0.975, or may be essentially 1.0. Also, the magnitude of stray capacitance of the RC circuit is typically small relative to the magnitude of the capacitance C. For example, the ratio of C to the total capacitance of the RC circuit may be at least about 0.9, at least about 0.95, at least about 0.975, or may be essentially 1.0. Together, resistance R and capacitance C typically at least essentially determine the RC time constant of RC circuit 12. For example, for R=150Ω and F=50 μF, RC=7.5 ms.

Resistor 14 may be formed as a resistive element disposed on, e.g., adhered to, a surface of the microfluidic device, e.g., to an external surface of the microfluidic device or to an internal surface a microfluidic network of the microfluidic device, e.g., to an internal surface of a channel, valve, or chamber thereof. Exemplary resistive elements include carbon ink-based resistive elements, silver ink-based resistive elements, metal oxide, e.g., silver oxide, resistive elements, and combinations thereof. Such resistive elements may be, e.g., printed or otherwise deposited on the diagnostic device. In some instances, the manufacturing process is imprecise so that the resistance of any one resistive element is unknown unless determined by measurement. However, the range of resistances resulting from such manufacturing processes may be empirically determined to fall between upper and lower bounds. Accordingly, it is possible to predict that any one manufactured resistive element will have a resistance greater than the minimum bound and less than the maximum bound.

Capacitor 16 is typically a non-polarized capacitor having a low temperature coefficient such that the capacitance exhibits low variation as a function of the temperature of the capacitor. To further reduce a temperature dependence of the capacitance, two or more capacitors may be combined, with a first one of the capacitors having a negative dependence on temperature and a second one of the capacitors having a positive dependence on temperature. In combination, such capacitors form a capacitive element with a combined capacitance with a reduced temperature dependence. Suitable capacitors, individually or in combination, may be formed, e.g., of polyester, polypropylene, ceramic, and combinations thereof. As used herein, the term capacitive element means an electronic element such as a capacitor or combination of capacitors having a suitable capacitance, polarization characteristics (e.g., being non-polarized), and low temperature coefficient.

DC power supply 22 includes a first power supply lead 24 and a second power supply lead 26. First power supply lead 24 bifurcates at location 25 into a first power supply sub-lead 24′, which terminates at a first power supply switch contact 42, and a first power supply sub-lead 24″, which terminates at a first power supply switch contact 36. Second power supply lead 26 bifurcates at location 27 into a second power supply sub-lead 26′, which terminates at a second power supply switch contact 32, and a second power supply sub-lead 26″, which terminates at a second power supply switch contact 46.

First RC circuit lead 18 bifurcates at a location 19 into a first RC circuit sub-lead 18′, which terminates at a first RC switch contact 44, and a first RC circuit sub-lead 18″, which terminates at a first RC switch contact 48. Second RC circuit lead 20 bifurcates at a location 21 into a second RC circuit sub-lead 20′, which terminates at a second RC switch contact 30, and a second RC circuit sub-lead 20″, which terminates at a second RC switch contact 34.

First switch 28 includes a bridging contact 38. In a first switch position as illustrated in FIG. 1, bridging contact 38 provides electrical connectivity between second RC switch contact 30 of second RC circuit sub-lead 20′ and second power supply switch contact 32 of second power supply sub-lead 26′. In a second switch position as illustrated by arrow 28′, bridging contact 38 provides electrical connectivity between second RC switch contact 34 of second RC circuit sub-lead 20″ and first power supply switch contact 36 of first power supply sub-lead 24″. Second switch 40 includes a bridging contact 50. In a first switch position as illustrated in FIG. 1, bridging contact 50 provides electrical connectivity between first RC switch contact 44 of first RC circuit sub-lead 18′ and first power supply switch contact 42 of first power supply sub-lead 24′. In a second switch position as illustrated by arrow 40′, bridging contact 50 provides electrical connectivity between first RC switch contact 48 of first RC circuit sub-lead 18″ and second power supply switch contact 46 of second power supply sub-lead 26″.

DC power supply 22 is configured to supply a constant electrical potential V_{PS} between location 25 of lead 24 and location 27 of lead 26. However, direction of the electrical potential drop across RC circuit 12 depends on the positions of first and second switches 28, 40. Referring also to FIG. 2, time-dependent electrical potential 200 illustrates the electrical potential drop across RC circuit 12 as would be observed with a voltmeter having its positive contact disposed at location 19 of first RC circuit lead 18 and its negative contact disposed at location 21 of second RC circuit lead 20. When first and second switches 28, 40 are each in the first switch position (FIG. 1), the electrical potential drop across RC circuit 12 so measured between first RC circuit lead 18 and second RC circuit lead 20 is V_{PS} (FIG. 2, during, e.g., a pulse 200′ between times τ₁ and τ₂). When first and second switches 28, 40 are each in the second switch position (as indicated by respective arrows 28′, 40′ in FIG. 1), the electrical potential drop so measured across RC circuit 12 between first RC circuit lead 18 and second RC circuit lead 20 is −V_{PS} (FIG. 2, during, e.g., a pulse 200″ between times τ₃ and τ₄). A pulse period τ_{P} is typically the same for each of the pulses. Between successive pulses, e.g., between time τ₂ (the end of pulse 200′) and time τ₃ (the start of pulse 200″), both switches 28, 40 are in an open position and the electrical potential drop across RC circuit 12 between first RC circuit lead 18 and second RC circuit lead 20 is 0. However, the open period is not illustrated in FIG. 2 because the duration of the open period is typically substantially smaller than the pulse period τ_{P}. For example, the ratio of the open period to the pulse period may be, e.g., about 0.01 or less, about 0.005 or less, or about 0.001 or less.

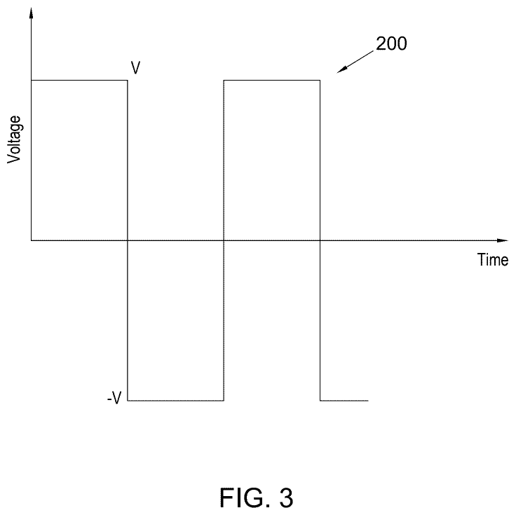

Although first and second switches 28, 40 permit the polarity of the electrical potential drop across RC circuit 12 to be reversed as shown in FIG. 2, the output of DC power supply 22 remains constant, i.e., V_{PS}. When DC power supply 22 is off, i.e., not generating any potential, the output is zero volts (FIG. 2, between times τ₀ and τ₁). Accordingly, the difference between the maximum potential and the minimum potential generated by DC power supply is V_{PS}. In contrast, as seen in FIG. 3, a zero-centered square-wave output 200 by an alternating current power supply (not shown) is not constant and instead oscillates between a maximum potential V_{AC} and a minimum potential −V_{AC}. The difference between the maximum potential V_{AC} and the minimum potential −V_{AC} output by such alternating current power supply is 2V_{AC}.

Heating system 10 may be configured and operated, e.g., as follows. Resistor 14 is positioned in thermal communication with location 52 to be heated. As discussed above, the RC time constant for RC circuit 12 is 7.5 ms for R=150Ω and F=50 μF. In addition to illustrating time dependent electrical potential 200 across RC circuit 12, FIG. 2 illustrates a time dependent electrical potential drop 202 across resistor 14, e.g., between location 19 of first circuit lead 18 and location 23 intermediate resistor 14 and capacitor 16, and a time dependent electrical charge of capacitor 16, e.g., between location 23 and location 21 of second circuit lead 20. Prior to initiating heating, DC power supply 22 is off so that the electrical potential drop across RC circuit 12 between first RC circuit lead 18 and second RC circuit lead 20 is 0 (time τ₀). At time τ₁, controller 11 actuates DC power supply 22 to a voltage V_{PS}, e.g., 12 V DC, positions switches 28, 40 in the first position (FIG. 1) and then maintains the switches in the first position for pulse period τ_{P} thereby generating voltage pulse 200′ (FIG. 2). A ratio of pulse period τ_{P} to the RC time constant (τ_{P}:RC) is 4 (τ_{P}=30 ms).

During pulse 200′, the electrical potential drop across resistor 14, as illustrated by a segment 202′ of time dependent electrical potential 202, decreases exponentially from V_{PS} at time τ₁ toward 0 V at time τ₂. Capacitor 16 charges exponentially from 0 V at time τ₁ toward V_{PS} at time τ₂ as illustrated by a segment 204′ of time dependent electrical potential 204. Because τ_{P} is long with respect to time constant RC, the potential drop across resistor 14 decreases essentially to 0 V and capacitor 16 charges essentially to V_{PS} by the end of pulse 200′ (time τ₂). For example, the potential drop across resistor 14 decreases to e\hat{ }(−τ_{P}/RC)×V_{PS}=0.018×V_{PS} and capacitor charges to an electrical potential given by [1−e\hat{ }(−τ_{P}/RC)×V_{PS}]=0.982×V_{PS}. The amount of energy dissipated by resistor 14 during pulse 200′ is 0.5×C×(0.98×V_{PS})²=3.45 mJ, which is essentially equal to 0.5×C×(V_{PS})²=3.6 mJ.

At time τ₂, controller 11 switches the switches 28, 40 from the first to the second position (as indicated by arrows 28′, 40′ in FIG. 1) thereby reversing the polarity of the electrical potential applied to RC circuit 12. The output of DC power supply 22 itself remains constant at V_{PS}. Controller 11 then maintains the switches in the second position for period τ_{P} generating a voltage pulse 200″ beginning at time τ₃ and ending at time τ₄ (FIG. 2). During the transition between pulses 200′ and 200″, the switches are briefly in the open position as discussed above. At time τ₃, the electrical potential drop across resistor 14, as illustrated by a segment 202″ of time dependent electrical potential 202, is given by the sum of the electrical potential of charged capacitor 16 and the electrical potential applied by DC power supply 22, i.e., essentially −2V_{PS}. During pulse 200″, the electrical potential drop across resistor 14 increases exponentially from essentially −2V_{PS} at time τ₃ toward 0 V at time τ₄. Capacitor 16 discharges exponentially from essentially V_{PS} at time τ₃ through 0 V at the midpoint of pulse 200″ toward −V_{PS} at time τ₄ as illustrated by a segment 204″ of time dependent electrical potential 204. By the end of pulse 200″ (time τ₄), the potential drop across resistor 14 increases essentially to 0 V, i.e., to −e\hat{ }(−τ_{P}/RC)×V_{PS}=−0.018×V_{PS} and capacitor 16 charges essentially to −V_{PS}, i.e., to an electrical potential given by −[1−e\hat{ }(−τ_{P}/RC)×V_{PS}]=−0.982×V_{PS}. The amount of energy dissipated by resistor 14 during pulse 200″ is 0.5×C×(2×0.98×V_{PS})²=13.8 mJ, which is essentially equal to 2×C×(V_{PS})²=14.4 mJ. The amount of energy dissipated during pulse 200″ is four times (4×) greater than the energy dissipated during the initial pulse 200′ because capacitor 16-—which was first charged to essentially V_{PS} during pulse 200′—combines with the electrical potential applied by DC power supply 22 to increase the electrical potential drop over and the resulting current flowing through resistor 14 during pulse 200″.

At time τ₄, controller 11 switches the switches 28, 40 from the second to the first position (FIG. 1) thereby reversing the polarity of the electrical potential applied to RC circuit 12. The output of DC power supply 22 itself remains constant at V_{PS}. Controller 11 then maintains the switches in the first position for period τ_{P} generating a voltage pulse 200′″ beginning at time τ₅ and ending at time τ₆ (FIG. 2). During the transition between pulses 200″ and 200′″, the switches are briefly in the open position as discussed above. At time τ₄, the electrical potential drop across resistor 14, as illustrated by a segment 202′″ of time dependent electrical potential 202, is given by the sum of the electrical potential on capacitor 16 and the electrical potential applied by DC power supply 22, i.e., essentially 2V_{PS}. During pulse 200′″, the electrical potential drop across resistor 14 decreases exponentially from 2V_{PS} at time τ₅ toward 0 V at time τ₆. Capacitor 16 discharges exponentially from essentially −V_{PS} at time τ₅ through 0 V at the midpoint of pulse 200′″ toward V_{PS} at time to as illustrated by a segment 204′″ of time dependent electrical potential 204. By the end of pulse 200′″ (time τ₆), the potential drop across resistor 14 has decreased essentially to 0 V, i.e., to e\hat{ }(−τ_{P}/RC)×V_{PS}=0.018×V_{PS} and capacitor 16 has charged essentially to V, i.e., to an electrical potential given by [1−e\hat{ }(−τ_{P}/RC)×V_{PS}]=0.982×V_{PS}. The amount of energy dissipated by resistor 14 during pulse 200′″ is the same as the energy dissipated during pulse 200″.

Following pulse 200′″ at time τ₇ controller 11 switches the switches 28, 40 from the first to the second position thereby again reversing the polarity of the electrical potential applied to RC circuit 12. Controller 11 then maintains the switches in the second position for period τ_{P} generating a voltage pulse 200″″ beginning at time τ₇ and ending at time τ₈ (FIG. 2). The behavior of the electrical potentials and energy dissipation during pulse 200″″ are the same as during pulse 200″. The process of switching the electrical potential applied to RC circuit 12 can be repeated for a number of times necessary to achieve a desired heating of object 52. For example, the electrical potential may be switched a number N times per second, wherein N may be between about (0.5×RC)⁻¹ and about (40×RC)¹, e.g., about (8×RC)⁻¹. In embodiments, for example, N may be between about 5 and 500, between about 10 and 200, or between about 15 and 100.

As discussed above, the average power dissipated by resistor 14 during initial pulse 200′ in which capacitor 16 first becomes charged depends on the square of the input potential: 0.5×C×(V_{PS})²/τ_{P}=0.12 W. In contrast, during each subsequent pulse, e.g., pulses 200″-200″″ and each pulse thereafter, the charged capacitor contributes to the electrical potential drop experienced by resistor 14. Therefore, the average power dissipated by resistor 14 during each subsequent pulse depends on the square of twice the input potential V_{PS}: 0.5×C×(2×V_{PS})²/τ_{P}=2×C×(V_{PS})²/τ_{P}=0.48 W. Heating system 10 efficiently converts the difference between the maximum and minimum electrical potential generated by DC power supply 22 (V_{PS}) to energy and power dissipated by resistor 14. The average power dissipated is inversely proportion to the duration of the period of each pulse as well as the number of pulses generated per second. However, the energy and power dissipated are independent of the resistance of resistor 14.

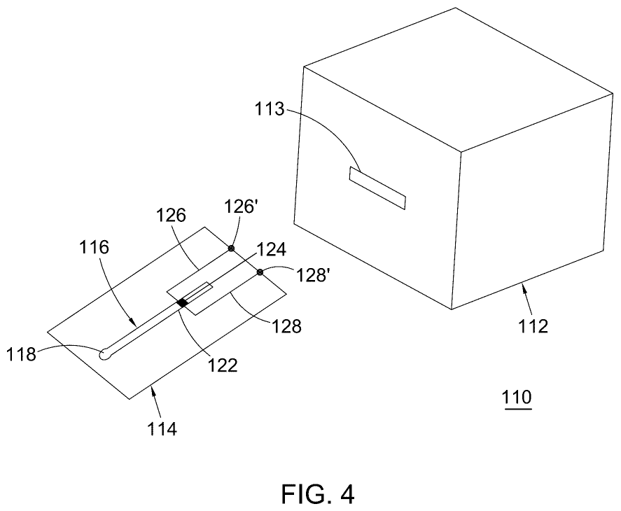

Referring to FIG. 4, a diagnostic reader 112 and a microfluidic device embodied as a microfluidic strip 114 is shown. Diagnostic reader 112 is configured to operate microfluidic strip 114 to perform a diagnostic assay for a target present in a liquid sample applied to the strip, e.g., as disclosed in the '933 application. Diagnostic strip 114 includes a microfluidic network 116 having a sample application zone 118 and a microchannel leading to a reaction zone 122, e.g., a detection zone. Microfluidic strip 114 may be configured and include features of microfluidic strips, e.g., reagents, gas chambers (gas bladders), and electrical sensing elements disclosed in the '933 application. Microfluidic strip 114 further includes a resistive element 124 disposed in thermal communication with reaction zone 122. For example, resistive element 124 may be formed from carbon ink-based resistive elements, silver ink-based resistive elements, metal oxide, e.g., silver oxide, resistive elements, and combinations thereof, printed or otherwise deposited on an external or internal surface of strip 114. Resistive element 124 is connected via a first lead 126 to a first terminal 126′ and by a second lead 128 to a second terminal 128′. When strip 114 is inserted into a receiving slot 113 of reader 112, terminals 126′, 128′ engage with corresponding connectors within the reader 112 providing electrical communication therebetween. Remaining portions of the heating system 10, e.g., a DC power supply, capacitor, and switching assemblies are housed within the reader 112. The resistive element 124 of the microfluidic strip 114 and the capacitor within the reader 112 form a series RC circuit as disclosed herein.

In use, strip 114 is inserted into receiving slot 113 and a liquid sample applied to application zone 118. The sample flows along microfluidic network 116 and is manipulated by reader 112 such as to move the liquid sample and mix the sample with reagents. The heating system 10 is actuated to heat reaction zone 122 to facilitate a desired reaction or condition of the liquid sample and reagents therein. The capacitance of the capacitor, the DC voltage of the power supply, and other operating conditions, e.g., pulse period and frequency, of the heating system 10 are chosen to produce a desired level of heating in the reaction zone 122. In some cases, the process for manufacturing such resistive elements leads to elements having a resistance that is not known with specificity. However, the range of resistances, e.g., with 99% confidence or more, can be determined. Typically, the duration τ_{P} of pulses generated by the heating system 10 are determined based on the largest likely resistance of such resistive element such that τ_{P}>RC time constant, e.g., so that the ratio τ_{P}:RC is between about 3 and 10, between about 3 and 7, between about 3 and 6, e.g., about 4.

In any of the embodiments employing a microfluidic device, e.g., microfluidic strip or cartridge, the capacitive element may be disposed on or within the microfluidic device. Typically in such embodiments, both the resistive element and capacitive element are disposed in series electrical communication to form the RC circuit on or within the microfluidic device. Such a microfluidic device typically includes at least one electrical contact corresponding to first RC circuit lead 18 and at least one electrical contact corresponding to second RC circuit lead 20. Each of the aforementioned electrical contacts may be configured, e.g., as a pair of contacts, e.g., as first RC circuit sub-lead 18′ and first RC circuit sub-lead 18″ on the one hand and as second RC circuit sub-lead 20′ and second RC circuit sub-lead 20″ on the other hand. A diagnostic reader configured to operate such a microfluidic device includes electrical contacts configured to engage with the electrical contacts of the microfluidic device. The diagnostic reader typically includes a power supply and switch system, e.g., including first and second switches, and a switch system controller as disclosed herein.

Techniques for preparing a capacitive element on or within a microfluidic device include, e.g., inkjet printing, screen printing, and 3D printing. Exemplary materials for forming such capacitive elements include (i) carbon-based nanomaterials such as activated carbon, carbon black, carbon nanotubes, graphene, reduced graphene oxide, and graphene oxide, (ii) ternary carbides and nitrides including one or more transition metal elements such as Ti₃C₂Tₓ, and (iii) hybrid materials such as materials including carbon based nanomaterials and metal oxides. Examples of capacitive elements (including suitable materials and fabrication techniques) that may be disposed on or within a microfluidic device, e.g., by printing, include those, e.g., disclosed in Advanced Materials, Volume 27, Issue 24, Jun. 24, 2015, pgs. 3669-3675; ACS Nano 2019, 13, 1, 54-60; Sci Rep 9, 13324 (2019); and Chem Phys Mater, Vol. 1, Issue 1, January 2022, Pages 17-38.

### EXAMPLE

A heating system was prepared to assess the capability of generating the same amount of heat from resistive elements having different resistances. A first test RC circuit was prepared by connecting a 27Ω resistor and a 47 μF non-polarized capacitor connected in series (RC=1.27 ms). A second test RC circuit was prepared by connecting a 150Ω resistor and a 47 μF non-polarized capacitor connected in series (RC=7.05 ms). Each resistor was a 1/8 W carbon film resistor. Each RC circuit included a first circuit lead corresponding to first circuit lead 18 of RC circuit 12 and a second circuit lead corresponding to second circuit lead 20 of RC circuit 12. The first circuit lead of each test RC circuit was connected to output 2 of a DRV8844PWP quad ½ H-bridge driver (“driver”) manufactured by Texas Instruments and the second circuit lead of each test RC circuit was connected to output 1 of the driver.

A constant (DC) power supply was used to input an electrical potential of 12 V to the driver. A switch system controller (an electronic processor) was used to input control signals to the driver thereby switching the state of the internal switches. In response to the control signals, the driver simultaneously alternated the polarity of the electrical potential (12 V) applied across the respective leads of each RC circuit as described for heating system 10. The pulse period τ_{P} was 28.2 ms, which is four times the length of the longest RC time constant of the two test RC circuits. The current and electrical potential capacities of the driver were sufficient to simultaneously operate each RC circuit without overloading the driver. Approximately 35 pulses per second were simultaneously delivered to each circuit. The temperature of each resistor was monitored by a respective thermocouple placed in thermal communication the resistor. The resistors were thermally isolated from one another, but exposed to the same ambient environment.

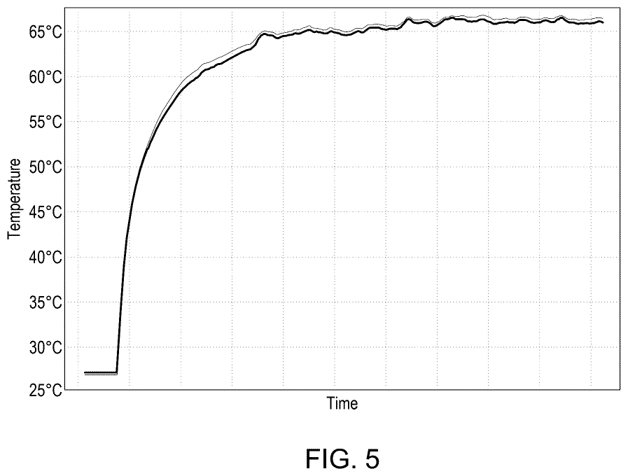

With reference to temperature vs time chart of FIG. 5, the temperature of each resistor increased from the ambient temperature (about 26° C.) to about 65° C. within about two minutes. At all times, the measured temperatures of the respective resistors are within about 1° C. of one another. The similar temperature profiles demonstrate the capability of the system to dissipate a constant average power from a resistor independently of its resistance. The ripples in the temperature vs time curves are believed to be due to air currents created by a computer cooling fan. After turning off the power to the heating system, the temperature of each resistor returned to ambient temperature within about 2.5 minutes. The respective ⅛ W resistors have a much larger thermal mass than ink-based resistive elements deposited on a surface of a diagnostic device. Accordingly, such diagnostic device resistive elements are expected to heat and cool much more rapidly than the ⅛ W test resistors employed in this Example.

Aspects of the disclosure are defined in the following numbered clauses:
1. A method of heating a reaction zone of a microfluidic strip, comprising:
   positioning a microfluidic strip in an operative position within a diagnostic reader, wherein the microfluidic strip comprises: (i) a microfluidic network comprising a reaction zone and (ii) a resistor adhered to a surface of the microfluidic strip in thermal communication with the reaction zone, wherein the resistor has a resistance R;
   connecting, in series, the resistor of the microfluidic strip and a capacitor disposed within the diagnostic reader to form an RC circuit having first and second RC circuit leads, wherein the capacitor has a capacitance C and the RC circuit has an RC time constant=TRC;
   preparing a reaction mixture within the reaction zone of the microfluidic strip, wherein the reaction mixture comprises a liquid sample and at least one reagent;
   generating and maintaining a constant electrical potential V_{PS} between first and second electrical supply leads disposed within the diagnostic reader; and
   while maintaining the constant electrical potential V_{PS} between the first and second supply leads, heating the reaction zone by alternatingly and repeatedly performing the following steps to thereby dissipate an average power 2×C×(V_{PS})²/τ_{P} over the resistor:
   (1) connecting the first supply lead to the first RC circuit lead and the second supply lead to the second RC circuit lead for a time period τ_{P} of about 4×τ_{RC}; and
   (2) connecting the second supply lead to the first RC circuit lead and the first supply lead to the second RC circuit lead for the time period τ_{P}.
2. The method of clause 1, comprising performing the step of heating the reaction zone for a period of between about 10 s and 6000 s.
3. The method of clause 1 or 2, wherein the step of heating the reaction zone comprises performing each of the steps of (1) connecting and (2) connecting between about 10 and 50 times per second.
4. The method of any of clauses 1-3, wherein V_{PS} is between about 5 and 25 volts, e.g., about 12 volts.
5. The method of any of clauses 1-4, wherein the resistance of the resistor is between about 20Ω and about 200Ω.
6. The method of any of clauses 1-5, wherein the capacitance of the capacitor is between about 20 μF and about 75 μF.
7. The method of any of clauses 1-6, comprising dissipating between an average power of between about 0.2 and about 1.0 W over the resistor.
8. The method of any of clauses 1-7, wherein the RC time constant is 7.5 ms, τ_{P}=30 ms, V_{PS}=12 V, and the average power dissipated is about 0.48 W.
9. The method of any of clauses 1-8, wherein the step of heating comprises heating the reaction mixture to a temperature of between about 40° C. and about 100° C.
10. The method of any of clauses 1-9, wherein a temperature of ambient gas surrounding the microfluidic strip is between about 20° C. and about 30° C. and the step of heating comprises heating the reaction mixture to a temperature of between about 20° C. and about 80° C. greater than the ambient temperature.
11. The method of any of clauses 1-10, wherein the resistor is a carbon ink-based resistor, a silver ink-based resistor, a metal oxide strip or a combination thereof.

All publications, patents, patent applications and other documents cited in this application are hereby incorporated by reference herein in their entireties for all purposes to the same extent as if each individual publication, patent, patent application or other document were individually indicated to be incorporated by reference for all purposes. While various specific embodiments have been illustrated and described, the above specification is not restrictive. It will be appreciated that various changes can be made without departing from the spirit and scope of the present disclosure(s). Many variations will become apparent to those skilled in the art upon review of this specification.

1. A method of heating at least one of a reaction zone, valve, or gas chamber of a microfluidic strip, comprising:
positioning a microfluidic strip in an operative position within a diagnostic reader, wherein the microfluidic strip comprises: (i) a microfluidic network comprising at least one of a reaction zone, a valve comprising a thermally responsive material, and a gas chamber configured to move liquid within the microfluidic network when the gas therein is heated and (ii) a resistor disposed in thermal communication with at least one of the at least one reaction zone, valve, or gas chamber, wherein the resistor has a resistance R;
connecting, in series, the resistor and a capacitor disposed on or within the microfluidic strip or within the diagnostic reader to form an RC circuit having first and second RC circuit leads, wherein the capacitor has a capacitance C and the RC circuit has an RC time constant=τ_{RC};
and
heating the at least one reaction zone, valve, or gas chamber in thermal communication with the resistor by alternatingly and repeatedly performing the following steps, wherein V_{PS} is an electrical potential between first and second electrical supply leads disposed within the diagnostic reader:
(1) connecting the first supply lead to the first RC circuit lead and the second supply lead to the second RC circuit lead for a time period τ_{P1} of between about 2 and about 5×τ_{RC}, while maintaining the electrical potential of about V_{PS} between the first and second supply leads during the time period τ_{P1}; and
(2) connecting the second supply lead to the first RC circuit lead and the first supply lead to the second RC circuit lead for a time period τ_{P2} of between about 2 and about 5×τ_{RC}, while maintaining the electrical potential of about V_{PS} between the first and second supply leads during the time period τ_{P2};
thereby dissipating an average power of about 2×C×(V_{PS})²/((τ_{P1}+τ_{P2})/2) over the resistor.

2. The method of claim 1, comprising performing the step of heating the reaction zone, valve, or gas chamber for a period of between about 10 s and 6000 s.

3. The method of claim 1, wherein the step of heating the reaction zone, valve, or gas chamber comprises performing each of the steps of (1) connecting and (2) connecting between about 10 and 50 times per second.

4. The method of claim 1, wherein V_{PS} is between about 5 and 25 volts, e.g., about 12 volts.

5. The method of claim 1, wherein the resistance R of the resistor is between about 20Ω and about 200Ω.

6. The method of claim 1, wherein the capacitance C of the capacitor is between about 20 μF and about 75 μF.

7. The method of claim 1, comprising dissipating between an average power of between about 0.2 and about 1.0 W over the resistor.

8. The method of claim 1, wherein the RC time constant is about 7.5 ms, τ_{P1} and τ_{P2} are each about 30 ms, V_{PS} is about 12 V, and the average power dissipated is about 0.48 W.

9. The method of claim 1, wherein the step of heating comprises heating the at least one reaction zone, valve, or gas chamber in thermal communication with the resistor to a temperature of between about 40° C. and about 100° C.

10. The method of claim 1, wherein an ambient temperature of gas surrounding the microfluidic strip is between about 20° C. and about 30° C. and the step of heating comprises heating the at least one reaction zone, valve, or gas chamber in thermal communication with the resistor to a temperature of between about 20° C. and about 80° C. greater than the ambient temperature.

11. The method of claim 1, wherein the resistor is a carbon ink-based resistor, a silver ink-based resistor, a metal oxide strip or a combination thereof.

12. The method of claim 1, wherein the capacitor is disposed within the diagnostic reader.

13. The method of claim 1, wherein the capacitor is disposed on or within the microfluidic strip.

14. The method of claim 1, wherein τ_{P1} and τ_{P2} are about the same.

15. The method of claim 1, wherein the thermally responsive material is a gas.

16. The method of 1, wherein the thermally responsive material is a wax, an oil, a hydrogel or a thermo-responsive polymer.

17. A system for heating at least one of a reaction zone, valve, or gas chamber of a microfluidic strip; the system comprising an RC circuit, a DC power supply operable to deliver a constant potential V_{PS}, and a switch system; wherein the system is configured to carry out the method of any preceding claim.
